(19) United States
(12) Patent Application Publication
(10) Pub. No.: US 2023/0311984 A1
(43) Pub. Date: Oct. 5, 2023

(54) DRIVING ABILITY DETERMINATION SYSTEM AND DRIVING ABILITY DETERMINATION METHOD

(71) Applicant: Honda Motor Co., Ltd., Tokyo (JP)

(72) Inventors: Taiki Iwama, Tokyo (JP); Toshifumi Matsuno, Tokyo (JP); Ryo Takebayashi, Tokyo (JP)

(21) Appl. No.: 18/126,956

(22) Filed: Mar. 27, 2023

(30) Foreign Application Priority Data
Mar. 31, 2022 (JP) 2022-058344

(51) Int. Cl. B62D 15/02; A61B 5/1171

(52) U.S. Cl. CPC B62D 15/0215; A61B 5/1171

(57) ABSTRACT

A driving ability determination system, includes: a processor and a memory coupled to the processor. The processor is configured to perform: acquiring time-series travel data of a vehicle; identifying a driver of the vehicle; and calculating an evaluation value indicating steering characteristics of the identified driver based on travel data during one cycle from a start time point to an end time point of the vehicle from among the acquired travel data.

### CROSS-REFERENCE TO RELATED APPLICATION

This application is based upon and claims the benefit of priority from Japanese Patent Application No. 2022-058344 filed on Mar. 31, 2022, the content of which is incorporated herein by reference.

### BACKGROUND OF THE INVENTION

### Field of the Invention

This invention relates to a driving ability determination system and a driving ability determination method configured to determine driving ability of a driver of a vehicle.

### Description of the Related Art

As this type of device, there has been conventionally known a device that measures a safe driving ability of a driver (see, for example, JP 2014-174848 A). In the device described in JP 2014-174848 A, a load is applied to a driver to disperse attention by intermittently outputting sound, steering entropy values indicating each of shaking of steering in a load state and a no-load state, are calculated, and a safe driving ability of the driver is evaluated based on a difference between shaking evaluation values calculated in the load state and the no-load state.

However, in the device described in JP 2014-174848 A, it is necessary to apply a load to a driver in order to evaluate a safe driving ability of the driver, which hinders driving.

### SUMMARY OF THE INVENTION

An aspect of the present invention is a driving ability determination system, including: a processor and a memory coupled to the processor. The processor is configured to perform: acquiring time-series travel data of a vehicle; identifying a driver of the vehicle; and calculating an evaluation value indicating steering characteristics of the identified driver based on travel data during one cycle from a start time point to an end time point of the vehicle from among the acquired travel data.

Another aspect of the present invention is a driving ability determination method, including the steps of: acquiring time-series travel data of a vehicle; identifying a driver of the vehicle; and calculating an evaluation value indicating steering characteristics of the identified driver based on travel data during one cycle from a start time point to an end time point of the vehicle from among the acquired travel data.

### BRIEF DESCRIPTION OF THE DRAWINGS

The objects, features, and advantages of the present invention will become clearer from the following description of embodiments in relation to the attached drawings, in which:

FIG. 1 is a diagram for explaining travel sections and driving loads;

FIG. 2 is a block diagram illustrating an example of a main configuration of a driving ability determination system according to an embodiment of the present invention;

FIG. 3 is a diagram for explaining a variation in steering angle of a vehicle;

FIG. 4 is a diagram exemplifying shaking of steering expressed in frequency;

FIG. 5A is a flowchart illustrating an example of a process executed by an arithmetic unit of FIG. 2;

FIG. 5B is a flowchart illustrating a modification of FIG. 5A when excluding travel data after a driver's seat door is opened;

FIG. 5C is a flowchart illustrating a modification of FIG. 5A when excluding travel data after the driver's seat door is opened and a driver leaves the seat;

FIG. 5D is a flowchart illustrating a modification of FIG. 5A when excluding travel data after the driver's seat door is opened and a seat door other than the driver's seat door is opened;

FIG. 5E is a flowchart illustrating a modification of FIG. 5A when excluding travel data after the driver's seat door is opened and a driver's seat belt is unbuckled; and

FIG. 5F is a flowchart illustrating a modification of FIG. 5A when excluding travel data after the driver's seat door is opened and the driver is switched.

### DETAILED DESCRIPTION OF THE INVENTION

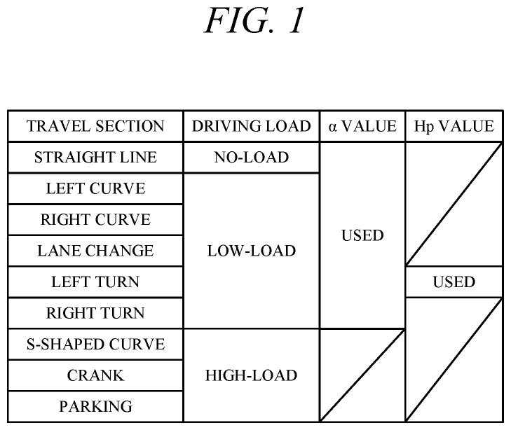

Hereinafter, embodiments of the present invention will be described with reference to FIGS. 1 to 5F. A driving ability determination system according to an embodiment of the present invention determines a driving ability of a driver of a vehicle. Generally, a driving behavior of the driver includes three elements of cognition, judgment, and operation. It is known that the ability related to a “cognitive function”, which is an intelligent function of a person related to the cognition and the judgment among the three elements, gradually decreases as the person gets older. The decrease in cognitive function makes it difficult to safely drive the vehicle.

Therefore, in the present embodiment, as will be described below, the driving ability determination system is configured to determine a driving ability, particularly a driving ability related to a cognitive function, based on travel data obtained when the driver drives the vehicle, such that the driver himself/herself or his/her family can support safe driving by grasping that the cognitive function of the driver tends to decrease.

FIG. 1 is a diagram for explaining travel sections and driving loads. As illustrated in FIG. 1, the travel sections in which a vehicle travels can be classified into a no-load section with almost no driving load applied to a driver by a driving behavior such as a straight line, a high-load section with a large driving load such as an S-shaped curve, a crank, or parking, and a low-load section with a middle driving load. More specifically, the no-load section is a travel section in which the driver is required to steer the vehicle in a small amount relative to a movement amount and a travel track of the vehicle forms a simple shape, and the high-load section is a section in which the driver is required to steer the vehicle in a large amount relative to a movement amount and a travel track of the vehicle forms a complicated shape. In such a high-load section, a high driving skill is required because steering is required very frequently, it is necessary to operate the steering in conjunction with the operation of an accelerator or a brake, and a sense of vehicle is also required. That is, a driver's driving skill greatly affects driving stability in the high-load section.

The low-load sections include travel sections such as a right curve, a left curve, a lane change, a right turn, and a left turn. Among these low-load sections, in a section in which the traveling direction of the vehicle is changed across the opposite lane at an intersection (a right turn section in a country or region where left-hand traffic is adopted for vehicles, or a left turn section in a country or region where right-hand traffic is adopted for vehicles, which will be simply referred to as a “left turn section”), when the driver recognizes a target track of the vehicle, it is necessary to grasp the situation of the opposite lane ahead and to grasp the situation of the target travel lane prior to the left turn. In this case, a shift of the line of sight between the opposite lane ahead and the target travel lane prior to the left turn increases the driver's mental activity, and a driving load, particularly a cognitive load related to cognition, increases as compared with those in the other low-load sections. For this reason, the cognitive function of the driver greatly affects driving stability in the left turn section. By evaluating driving stability based on such travel data obtained in the left turn section, it is possible to determine a driving ability related to the cognitive function of the driver.

Examples of the travel section in which the cognitive function of the driver affects driving stability include, in addition to the left turn section, a section in which the number of times the line of sight shifts is larger than a predetermined number such as a section in which many signs are provided or a two-way traffic section, a section in which traffic lights are provided, a section in which the number of pedestrians is larger than a predetermined number such as a downtown, a section in which the number of blind spots during driving is larger than a predetermined number such as an intersection with poor visibility, a section in which a plurality of roads intersect, and the like. Therefore, the driving ability related to the cognitive function of the driver can also be determined by acquiring travel data in such a section in such a manner as to be distinguishable from that in the other sections and evaluating driving stability based on the acquired travel data. When time-series position information is acquired in addition to the travel data of the vehicle, a specific preset travel section can be identified based on the position information.

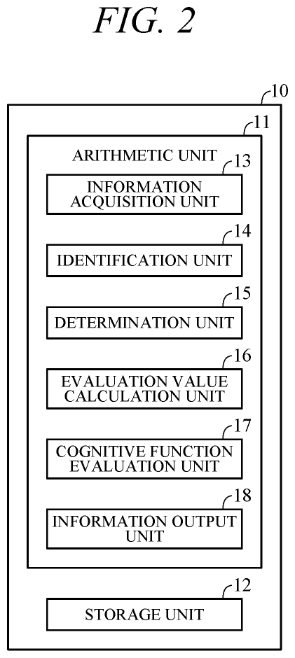

FIG. 2 is a block diagram illustrating an example of a main configuration of the driving ability determination system (hereinafter, the system) 10. As illustrated in FIG. 2, the system 10 includes a computer having an arithmetic unit 11 such as a CPU, a storage unit 12 such as a ROM or a RAM, and peripheral circuits thereof. The arithmetic unit 11 includes, as functional components, an information acquisition unit 13, an identification unit 14, a determination unit 15, an evaluation value calculation unit 16, a cognitive function evaluation unit 17, and an information output unit 18. The storage unit 12 stores programs to be executed by the arithmetic unit 11 and information such as setting values. The system 10 may be configured as an in-vehicle device mounted on a vehicle, or may be configured as a server device or the like provided outside the vehicle.

The information acquisition unit 13 acquires time-series travel data of a vehicle for each pre-registered driver. For example, the information acquisition unit 13 acquires travel data measured in a pre-registered vehicle that is routinely driven by each driver. The travel data includes information on a steering angle of a steering wheel detected by a sensor mounted on each vehicle, information on whether each seat door including a driver's seat door is opened, information on whether a driver's seat is occupied, information on whether a driver's seat belt is buckled, information on a biologically authenticated driver, and the like.

The travel data of each vehicle, more specifically, the travel data for each cycle from a start time point to an end time point of each vehicle, is transmitted to the system 10, for example, at a predetermined cycle via a telematics control unit (TCU) mounted on the vehicle together with a vehicle ID given in advance to each vehicle. The information acquisition unit 13 acquires travel data transmitted from a pre-registered vehicle as time-series travel data for each pre-registered driver. The time-series travel data for each driver acquired by the information acquisition unit 13 is stored in the storage unit 12.

Here, one cycle refers to a period from a start time point to an end time point of the vehicle as a use period of the vehicle for movement from a departure point to a destination point. The start time point of the vehicle may be, for example, a time point when the ignition of the vehicle is turned on, and the end time point of the vehicle may be, for example, a time point when the ignition of the vehicle is turned off.

An input/output unit including an output unit such as a display and a speaker and an input unit such as a touch panel and a microphone is provided in the vicinity of the driver's seat of each vehicle. When the vehicle is activated, a current driver who drives the vehicle is identified via the input/output unit. For example, when a user ID (user name) of a pre-registered driver is notified via the output unit, the user is urged to give confirmation or input a password, and the user inputs a confirmation result via the input unit, the current driver is identified according to the confirmation result. Instead of or in addition to the input/output device, the identification unit may include a camera that performs face authentication or iris authentication, and a sensor that performs fingerprint authentication, palm print authentication, finger vein authentication, or the like. In addition, voice authentication may be performed via the microphone. Information on the driver identified by the identification unit at the time of starting the vehicle is also transmitted to the system 10 as travel data.

Based on the user information registered in advance to use a driving ability determination service, the identification unit 14 acquires travel data and identifies a driver to be evaluated for driving stability. More specifically, the identification unit 14 identifies driver information such as a vehicle ID of the pre-registered vehicle that is routinely driven by the driver to be evaluated or a user ID of the driver. The driver information identified by the identification unit 14 is stored in the storage unit 12. The travel data acquired by the information acquisition unit 13 from each vehicle is associated with each driver based on the driver information, and is accumulated in the storage unit 12 as travel data for each driver.

The determination unit 15 determines whether the driver's seat door has been opened during one cycle from the start to the end of the vehicle based on the travel data for each driver stored in the storage unit 12. That is, the determination unit 15 determines whether there is a possibility that the driver's seat door has been opened to switch the driver during one cycle. When the determination unit 15 determines that there is a possibility that the driver has been switched during one cycle, travel data obtained thereafter may be excluded from the travel data for each driver stored in the storage unit 12.

The determination unit 15 may determine whether or not there is a possibility that the driver has been switched during one cycle by determining whether or not the driver's seat door has been opened and the driver has left the seat. The determination unit 15 may determine whether there is a possibility that the driver has been switched during one cycle by determining whether the driver's seat door has been opened and a seat door other than the driver's seat door has been opened. The determination unit 15 may determine whether there is a possibility that the driver has been switched during one cycle by determining whether the driver's seat door has been opened and the driver's seat belt has been unbuckled.

When the driver is identified by the input/output unit on the vehicle side at all times or in conjunction with whether the driver's seat door is opened, the determination unit 15 can directly determine whether the driver has been switched based on a driver identifying result. In this case, the determination unit 15 may determine whether the driver's seat door has been opened and the driver has been switched during one cycle.

The evaluation value calculation unit 16 calculates an a value and an Hp value indicating steering characteristics of an individual driver based on the travel data for each driver stored in the storage unit 12. More specifically, the evaluation value calculation unit 16 determines a travel section for each unit time based on a time-dependent change in steering angle, and calculates an a value indicating steering characteristics of the driver based on travel data obtained in a period in which it is determined that the vehicle is traveling in a no-load section or a low-load section (no-load/low-load section). In addition, the evaluation value calculation unit 16 calculates an Hp value indicating steering characteristics of the driver, when the cognitive load increases based on travel data obtained in a period in which it is determined that the vehicle is traveling in a left turn section.

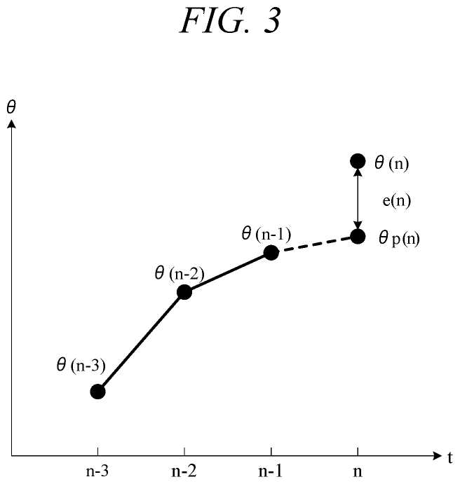

FIG. 3 is a diagram for explaining a variation in steering angle θ of the vehicle. When the vehicle is driving in a stable state, the steering is smoothly performed without shaking, and a variation in steering angle θ becomes small. On the other hand, when the vehicle is driving in an unstable state, steering is shaking, and a variation in steering angle θ increases.

More specifically, as illustrated in FIG. 3, based on actual steering angles θ(n−3), θ(n−2), and θ(n−1) at time points n−3, n−2, and n−1 immediately before a specific time point n, a predicted steering angle θp(n) at the time point n is calculated by second-order Taylor expansion with respect to the time point (n−1). Since the predicted steering angle θp(n) is a value estimated on the assumption that the steering is performed smoothly, the predicted steering angle θp(n) matches an actual steering angle θ(n) when the actual steering is performed smoothly, and deviates from the actual steering angle θ(n) according to a degree of shaking when the actual steering is shaking. Such a degree of shaking can be expressed as a predicted error e(n) calculated by the following equation (i).

e(n)=θ(n)−θp(n) (i)

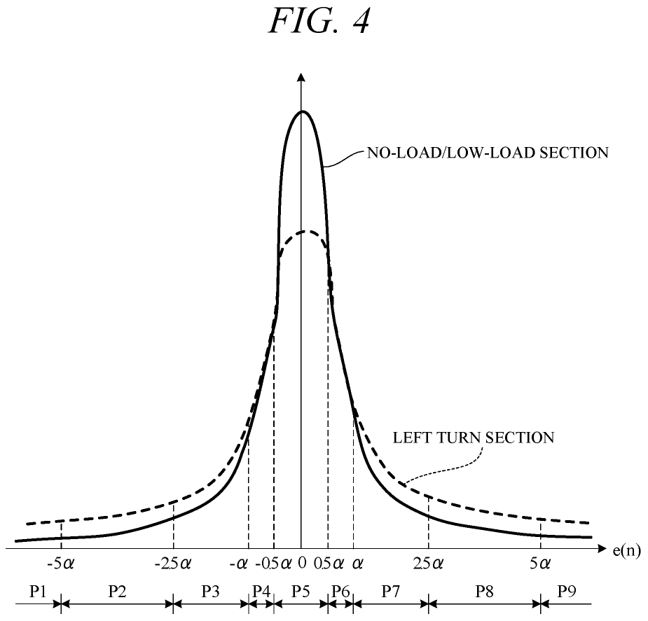

FIG. 4 is a diagram exemplifying shaking of steering expressed in frequency, and illustrates an example of a predicted error e(n) expressed in frequency. The evaluation value calculation unit 16 calculates a predicted steering angle θp(n) and a predicted error e(n) at each time point n based on the travel data in the no-load/low-load section, and calculates a 90th percentile value (a value) in a frequency distribution of the predicted error e(n) as indicated by the solid line. As the steering is smoother and the shaking of steering is smaller, the frequency distribution of the predicted error e(n) has a sharper shape with respect to “0°” with no shaking of steering, and the a value becomes smaller. On the other hand, as the shaking of steering is larger, the frequency distribution of the predicted error e(n) has a broader shape and the a value becomes larger.

By using the travel data in the no-load/low-load section excluding the high-load section in which a large amount of steering is required and a driving skill greatly affects shaking of steering, the a value indicating shaking of steering of the driver in the normal state can be appropriately calculated.

Furthermore, the evaluation value calculation unit 16 calculates an Hp value indicating steering characteristics of the driver when the cognitive load increases, based on the calculated α value and the travel data in the left turn section. More specifically, the evaluation value calculation unit 16 calculates a predicted steering angle θp(n) and a predicted error e(n) at each time point n based on the travel data in the left turn section, and a frequency distribution of the predicted error e(n) as indicated by the broken line is divided into nine ranges P1 to P9 based on a values. That is, based on eight reference values −5α, −2.5α, −α, −0.5α, 0.5α, α, 2.5α, and 5α, the frequency distribution of the predicted error e(n) is divided into nine ranges P1 (−5α or less), P2 (−5α to −2.5α), P3 (−2.5α to −α), P4 (−α to −0.5α), P5 (−0.5α to 0.5α), P6 (0.5α to α), P7 (α to 2.5α), P8 (2.5α to 5α), and P9 (5α or more). Then, a steering entropy value (Hp value) is calculated by the following equation (ii) based on the ratios p1 to p9 of the ranges P1 to P9.

Hp=−Σpi·log₉pi (ii)

The Hp value, which indicates smoothness of steering, becomes a smaller value as the frequency distribution of the predicted error e(n) becomes sharper with less shaking of steering, and becomes a larger value as the frequency distribution of the predicted error e(n) becomes broader with more shaking of steering. By using the travel data in the left turn section where the line of sight shifts a large number of times and the cognitive function greatly affects shaking of steering, it is possible to appropriately calculate an Hp value indicating shaking of steering of the driver when the cognitive load becomes higher as compared with that in the normal state.

The cognitive function evaluation unit 17 evaluates a cognitive function of the driver based on the Hp value calculated by the evaluation value calculation unit 16. That is, by continuously monitoring the Hp value indicating shaking of steering when the cognitive load increases, it is possible to evaluate that the cognitive function of the driver tends to decrease. For example, when the Hp value calculated periodically (e.g., monthly) based on the travel data on routine driving tends to increase, it is evaluated that the cognitive function tends to decrease.

The information output unit 18 transmits a result of the evaluation by the cognitive function evaluation unit 17 to a user terminal of the driver himself/herself, his/her family, or the like. For example, a notification can be transmitted to an e-mail address registered in advance. In this case, with the notification as a trigger, the driver himself/herself, his/her family, or the like can consider return of the driver's license, replacement with a vehicle having enhanced driving support functions, or the like. Since objective information is provided based on the travel data, it is easy for the driver to accept the current state of his/her cognitive function, and the driver can consider an appropriate response at an early stage.

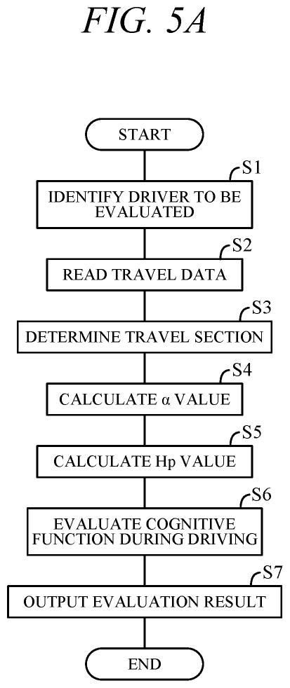

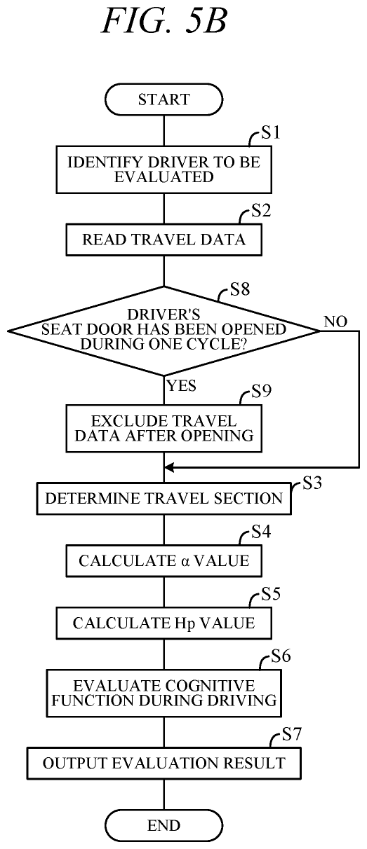

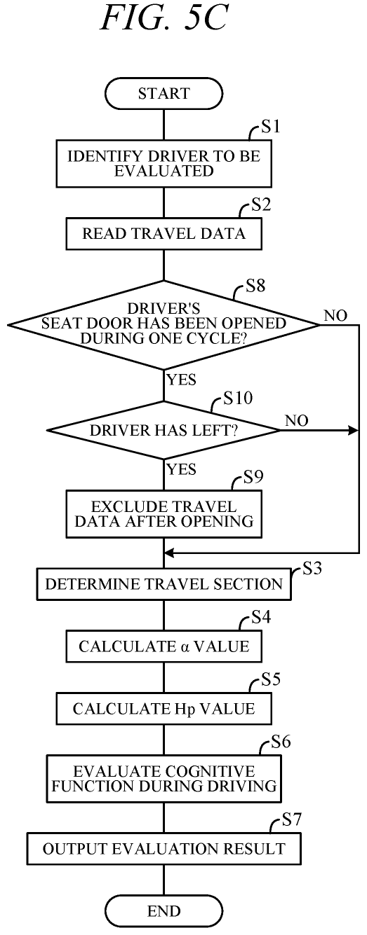

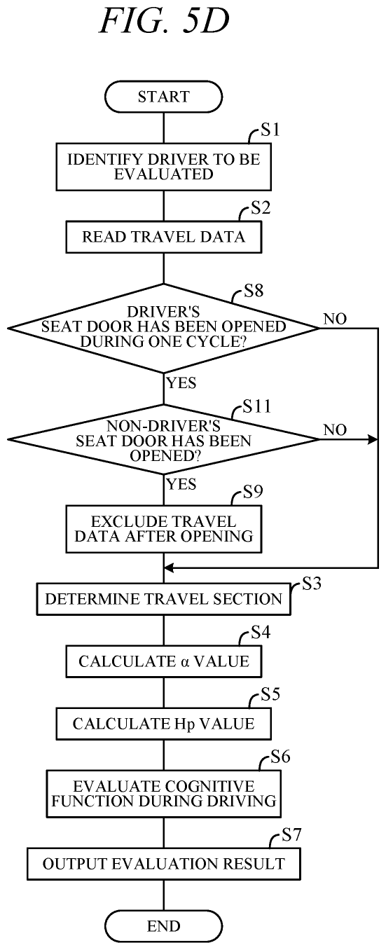

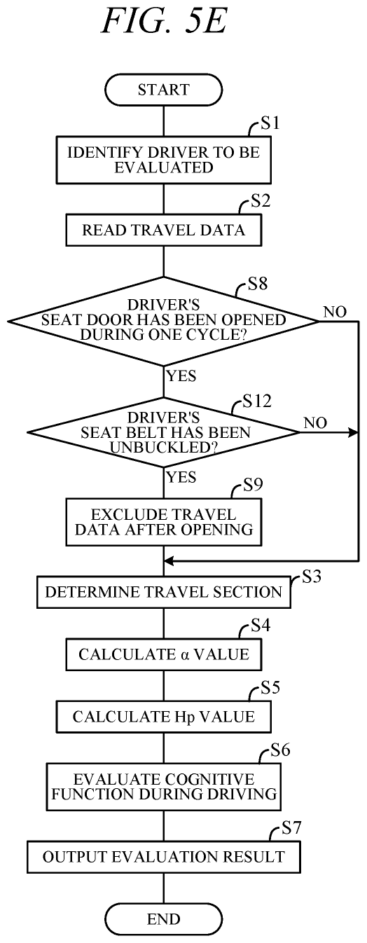

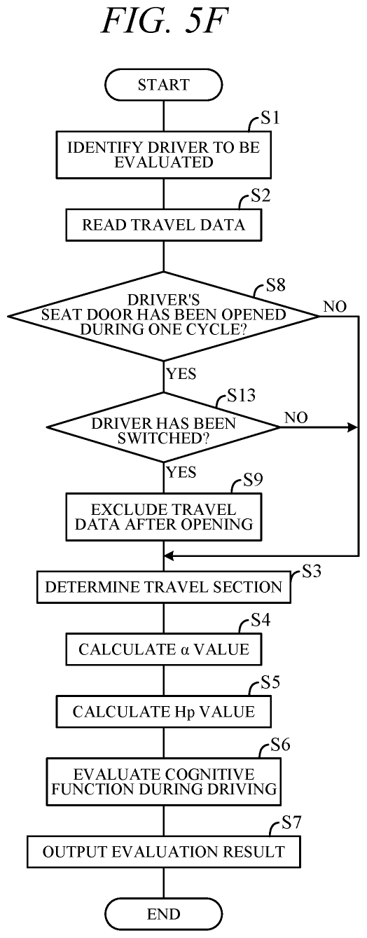

FIG. 5A is a flowchart illustrating an example of a process executed by the arithmetic unit 11 of the system 10, and illustrates an example of a process of evaluating a driving ability of a pre-registered driver based on all travel data of a pre-registered vehicle. The process illustrated in this flowchart is executed, for example, periodically. First, in S1 (S: a step of the process), a driver (user ID) to be evaluated is identified. Next, in S2, travel data associated with the user ID identified in S1 is read from the storage unit 12. Next, in S3, a travel section for each unit time is determined based on the travel data read in S2.

Next, in S4, an a value is calculated based on travel data in a period determined as a no-load/low-load section in S3 among the travel data read in S2. Next, in S5, an Hp value is calculated based on travel data in a period determined as a left turn section in S3 among the travel data read in S2 and the a value calculated in S4. The latest Hp value calculated in S5 is stored and accumulated in the storage unit 12. Next, in S6, the latest Hp value stored in the storage unit 12 is compared with the past Hp values to determine a driving ability related to the cognitive function of the driver. Next, in S7, an evaluation result of S6 is transmitted to a mail address registered in advance, and the process ends.

As described above, since the a value and the Hp value, which are indices for determining a driving ability of the driver, can be calculated only based on the routine travel data, the driving ability can be determined without hindering the driving (S1 to S5). In addition, since the a value and the Hp value are calculated based on the travel data during one cycle from the start to the end of the pre-registered vehicle, the driving ability of the individual pre-registered driver can be appropriately determined (S2 to S6). In addition, since the cognitive function of the driver is automatically evaluated only based on the route travel data, and the evaluation result is notified to the driver himself/herself or his/her family, it is possible to reduce the watching burden on the family living apart from the elderly person who drives the vehicle (S1 to S7).

Each of FIGS. 5B to 5F is a flowchart illustrating a modification of FIG. 5A, and FIG. 5B is a flowchart illustrating an example of a process in a case where a driving ability of each driver is evaluated after excluding travel data after the driver's seat door is opened from the travel data during each cycle. In this case, after the travel data for each driver stored in the storage unit is read in S2, the process proceeds to S8, and it is determined whether the driver's seat door has been opened during each cycle based on information on whether each seat door has been opened, which is included in the travel data.

If YES in S8, the process proceeds to S9, and the travel data after the driver's seat door is opened among the travel data during each cycle is excluded from the travel data for each driver stored in the storage unit. Then, the process proceeds to S3. If NO in S8, the process proceeds directly to S3. As a result, when the driver's seat door has been opened during each cycle and there is a possibility that the driver has been switched, travel data obtained thereafter is excluded from the travel data for each driver, thereby more appropriately determining a driving ability of each driver.

FIG. 5C is a flowchart illustrating an example of a process in a case where a driving ability of each driver is evaluated after excluding travel data after the driver's seat door is opened and the driver leaves the seat from the travel data of each cycle. In this case, after the travel data for each driver stored in the storage unit 12 is read in S2, it is determined in S8 whether the driver's seat door has been opened during each cycle. If YES, the process proceeds to S10. In S10, based on information on whether the driver's seat is occupied, which is included in the travel data, it is determined whether the driver has left the seat when the driver's seat door was opened during each cycle.

If YES in S10, the process proceeds to S9, and the travel data after the driver's seat door is opened among the travel data during each cycle is excluded from the travel data for each driver stored in the storage unit. Then, the process proceeds to S3. If NO in S8 or S10, the process proceeds directly to S3. As a result, only when there is a high possibility that the driver leaves the seat and the driver is switched during each cycle, travel data obtained thereafter is excluded from the travel data for each driver, thereby more appropriately determining a driving ability of each driver while effectively using the travel data.

FIG. 5D is a flowchart illustrating an example of a process in a case where a driving ability of each driver is evaluated after excluding travel data after the driver's seat door is opened and a seat door other than the driver's seat door is opened from the travel data of each cycle. In this case, after the travel data for each driver stored in the storage unit 12 is read in S2, it is determined in S8 whether the driver's seat door has been opened during each cycle. If YES, the process proceeds to S11. In S11, based on information on whether each seat door is opened, which is included in the travel data, it is determined whether a seat door other than the driver's seat door is opened when the driver's seat door is opened during each cycle.

If YES in S11, the process proceeds to S9, and the travel data after the driver's seat door is opened among the travel data during each cycle is excluded from the travel data for each driver stored in the storage unit. Then, the process proceeds to S3. If NO in S8 or S11, the process proceeds directly to S3. As a result, only when the driver's seat door and another seat door are simultaneously opened during each cycle and there is a high possibility that the driver and another occupant are switched to each other, travel data obtained thereafter is excluded from the travel data for each driver. Therefore, it is possible to more appropriately determine a driving ability of each driver while effectively using the travel data.

FIG. 5E is a flowchart illustrating an example of a process in a case where a driving ability of each driver is evaluated after excluding travel data after the driver's seat door is opened and the driver's seat belt is unbuckled from the travel data of each cycle. In this case, after the travel data for each driver stored in the storage unit 12 is read in S2, it is determined in S8 whether the driver's seat door has been opened during each cycle. If YES, the process proceeds to S12. In S12, based on information on whether the driver's seat belt is buckled, which is included in the travel data, it is determined whether the driver's seat belt has been unbuckled when the driver's seat door was opened during each cycle.

If YES in S12, the process proceeds to S9, and the travel data after the driver's seat door is opened among the travel data during each cycle is excluded from the travel data for each driver stored in the storage unit. Then, the process proceeds to S3. If NO in S8 or S12, the process proceeds directly to S3. Only when there is a high possibility that the driver unbuckles the seat belt and the driver is switched during each cycle, travel data obtained thereafter is excluded from the travel data for each driver, thereby more appropriately determining a driving ability of each driver while effectively using the travel data.

FIG. 5F is a flowchart illustrating an example of a process in a case where a driving ability of each driver is evaluated after excluding travel data after the driver's seat door is opened and the driver is switched from the travel data of each cycle. In this case, after the travel data for each driver stored in the storage unit 12 is read in S2, it is determined in S8 whether the driver's seat door has been opened during each cycle. If YES, the process proceeds to S13. In S13, based on a driver identifying result, which is included in the travel data, it is determined whether or not the driver has been switched when the driver's seat door was opened during each cycle.

If YES in S13, the process proceeds to S9, and the travel data after the driver's seat door is opened among the travel data during each cycle is excluded from the travel data for each driver stored in the storage unit. Then, the process proceeds to S3. If NO in S8 or S13, the process proceeds directly to S3. As a result, only when the driver is switched during each cycle, travel data obtained thereafter is excluded from the travel data for each driver, thereby more appropriately determining a driving ability of each driver while effectively using the travel data.

According to the present embodiment, the following operational effects can be achieved.

(1) A system 10 includes: an information acquisition unit 13 that acquires time-series travel data of a vehicle, an identification unit 14 that identifies a driver of the vehicle, and an evaluation value calculation unit 16 that calculates an a value and an Hp value indicating steering characteristics of the driver identified by the identification unit 14 based on travel data during one cycle from a start time point to an end time point of the vehicle among the travel data acquired by the information acquisition unit 13 (FIGS. 2 and 5A). As a result, since the a value and the Hp value, which are indices for determining a driving ability of the driver, can be calculated based on the routine travel data, the driving ability can be determined without hindering the driving. In addition, since the a value and the Hp value are calculated based on the travel data during one cycle from the start to the end of the pre-registered vehicle, the driving ability of the individual pre-registered driver can be appropriately determined.

(2) The evaluation value calculation unit 16 calculates an a value and an Hp value indicating steering characteristics of the driver at a start time point of a vehicle identified by the identification unit 14 based on travel data during one cycle (FIGS. 5A to 5F). That is, the a value and the Hp value of an individual driver identified at the time of starting a pre-registered vehicle are calculated based on the travel data during one cycle from the start to the end of the vehicle that is routinely driven by the driver to be evaluated. Therefore, it is possible to more appropriately determine a driving ability of the individual driver to be evaluated.

(3) The system 10 further includes a storage unit 12 that stores the travel data acquired by the information acquisition unit 13 and the a value and the Hp value calculated by the evaluation value calculation unit 16 in association with the driver at the start time point of the vehicle identified by the identification unit 14 (FIG. 2). With such a configuration, the travel data acquired by the information acquisition unit 13 from each vehicle and the evaluation values calculated by the evaluation value calculation unit 16 are accumulated in association with each pre-registered driver in the storage unit 12 as travel data for each driver to be evaluated.

(4) The travel data includes information on a state of the vehicle detected by a sensor mounted on the vehicle. The system 10 further includes a determination unit 15 that determines whether the driver has been switched during one cycle based on the travel data acquired by the information acquisition unit 13 (FIG. 2). The evaluation value calculation unit 16 specifies travel data from the start time point to a time point at which the determination unit 15 determines that the driver has been switched among the travel data during one cycle, and calculates an a value and an Hp value based on the specified travel data (FIGS. 5B to 5F). As a result, when there is a possibility that the driver has been switched, travel data obtained thereafter can be excluded from the travel data for the individual pre-registered driver, thereby more appropriately determining a driving ability of the individual driver.

(5) The travel data includes information on whether a driver's seat door is opened as the information on the state of the vehicle. The determination unit 15 determines whether the driver has been switched during one cycle by determining whether the driver's seat door has been opened during one cycle based on the travel data acquired by the information acquisition unit 13 (FIG. 5B). When the driver's seat door has been opened between the start and the end of the vehicle and there is a possibility that the driver has been switched, travel data obtained thereafter is excluded from the travel data for the individual pre-registered driver. Therefore, it is possible to more appropriately determine a driving ability of the individual driver.

(6) The travel data includes information on whether the driver's seat door is opened and information on whether a driver's seat is occupied as the information on the state of the vehicle. Based on the travel data acquired by the information acquisition unit 13, the determination unit 15 determines whether the driver has been switched during one cycle, by determining whether the driver's seat door has been opened and the driver has left the seat during one cycle (FIG. 5C). Only when the driver's seat door has been opened between the start and the end of the vehicle and the driver has left the seat, and there is a possibility that the driver has been switched, travel data obtained thereafter is excluded. Therefore, it is possible to appropriately determine a driving ability of the individual driver while effectively using the travel data.

(7) The travel data includes information on whether the driver's seat door is opened and information on whether a seat door other than the driver's seat door is opened as the information on the state of the vehicle. Based on the travel data acquired by the information acquisition unit 13, the determination unit 15 determines whether the driver has been switched during one cycle, by determining whether the driver's seat door has been opened and a seat door other than the driver's seat door has been opened during one cycle (FIG. 5D). Only when the driver's seat door and another seat door have been simultaneously opened between the start and the end of the vehicle and there is a possibility that the driver and another occupant have been switched to each other, travel data obtained thereafter is excluded. Therefore, it is possible to appropriately determine a driving ability of the individual driver while effectively using the travel data.

(8) The travel data includes information on whether the driver's seat door is opened and information on whether a driver's seat belt is buckled as the information on the state of the vehicle. Based on the travel data acquired by the information acquisition unit 13, the determination unit 15 determines whether the driver has been switched during one cycle, by determining whether the driver's seat door has been opened and the driver's seat belt has been unbuckled during one cycle (FIG. 5E). Only when the driver's seat door has been opened between the start and the end of the vehicle and the driver's seat belt has been unbuckled, and there is a possibility that the driver has been switched, travel data obtained thereafter is excluded. Therefore, it is possible to appropriately determine a driving ability of the individual driver while effectively using the travel data.

(9) The travel data includes information on whether the driver's seat door is opened and information on a biologically authenticated driver as the information on the state of the vehicle. Based on the travel data acquired by the information acquisition unit 13, the determination unit 15 determines whether the driver has been switched during one cycle, by determining whether the driver's seat door has been opened and the driver has been switched during one cycle (FIG. 5F). Only when the driver's seat door has been opened between the start and the end of the vehicle and the driver has been switched, travel data obtained thereafter is excluded. Therefore, it is possible to appropriately determine a driving ability of the individual driver while effectively using the travel data.

In the above-described embodiment, it has been described as an example, with reference to FIG. 1, etc. that an a value is calculated based on travel data when the vehicle travels in a no-load/low-load section and an Hp value is calculated based on travel data when the vehicle travels in a left turn section, but the evaluation value calculation unit is not limited thereto. Any evaluation value calculation unit may be used as long as it calculates an evaluation value indicating steering characteristics of each driver based on travel data for each driver.

Although the present invention has been described above as a driving ability determination system, the present invention can also be used as a driving ability determination method. That is, the driving ability determination method includes: an information acquisition step S2 of acquiring time-series travel data of a vehicle; an identification step S1 of identifying a driver of the vehicle; and evaluation value calculation steps S4 and S5 of calculating evaluation values indicating steering characteristics of the driver identified in the identification step S1 based on the travel data during one cycle from a start time point to an end time point of the vehicle among the travel data acquired in the information acquisition step S2 (FIG. 5A).

The above embodiment can be combined as desired with one or more of the aforesaid modifications. The modifications can also be combined with one another.

According to the present invention, it is possible to determine the driving ability without hindering the driving.

Above, while the present invention has been described with reference to the preferred embodiments thereof, it will be understood, by those skilled in the art, that various changes and modifications may be made thereto without departing from the scope of the appended claims.

1. A driving ability determination system, comprising:
a processor and a memory coupled to the processor, wherein
the processor is configured to perform:
acquiring time-series travel data of a vehicle;
identifying a driver of the vehicle; and
calculating an evaluation value indicating steering characteristics of the identified driver based on travel data during one cycle from a start time point to an end time point of the vehicle from among the acquired travel data.

2. The driving ability determination system according to claim 1, wherein
the processor is configured to perform:
the calculating including calculating the evaluation value of the driver identified at the start time point based on the travel data during the one cycle.

3. The driving ability determination system according to claim 2, wherein
the memory is configured to store the acquired travel data and the calculated evaluation value in association with the driver identified at the start time point.

4. The driving ability determination system according to claim 1, wherein
the travel data includes information on a state of the vehicle detected by a sensor mounted on the vehicle, wherein
the processor is configured to perform:
determining whether the driver has been switched during the one cycle based on the acquired travel data;
specifying travel data from the start time point to a time point at which it is determined that the driver has been switched from among the travel data during the one cycle; and
the calculating including calculating the evaluation value based on the specified travel data.

5. The driving ability determination system according to claim 4, wherein
the travel data includes information on whether a driver's seat door is opened as the information on the state of the vehicle, wherein
the processor is configured to perform:
determining whether the driver has been switched during the one cycle by determining whether the driver's seat door has been opened during the one cycle based on the acquired travel data.

6. The driving ability determination system according to claim 4, wherein
the travel data includes: information on whether a driver's seat door is opened; and information on whether a driver's seat is occupied, as the information on the state of the vehicle, wherein
the processor is configured to perform:
determining whether the driver has been switched during the one cycle by determining whether the driver's seat door has been opened and the driver has left the driver's seat during the one cycle based on the acquired travel data.

7. The driving ability determination system according to claim 4, wherein
the travel data includes: information on whether a driver's seat door is opened; and information on whether a seat door other than the driver's seat door is opened, as the information on the state of the vehicle, wherein
the processor is configured to perform:
determining whether the driver has been switched during the one cycle by determining whether the driver's seat door has been opened and the seat door other than the driver's seat door has been opened during the one cycle based on the acquired travel data.

8. The driving ability determination system according to claim 4, wherein
the travel data includes: information on whether a driver's seat door is opened; and information on whether a driver's seat belt is buckled, as the information on the state of the vehicle, wherein
the processor is configured to perform:
determining whether the driver has been switched during the one cycle by determining whether the driver's seat door has been opened and the driver's seat belt has been unbuckled during the one cycle based on the acquired travel data.

9. The driving ability determination system according to claim 4, wherein
the travel data includes: information on whether a driver's seat door is opened; and information on a biologically authenticated driver, as the information on the state of the vehicle, wherein
the processor is configured to perform:
determining whether the driver has been switched during the one cycle by determining whether the driver's seat door has been opened and the biologically authenticated driver has been switched during the one cycle based on the acquired travel data.

10. The driving ability determination system according to claim 1, wherein
the travel data includes information on a steering angle of a steering wheel detected by a sensor mounted on the vehicle, and is transmitted to the driving ability determination system through a telematics control unit mounted on the vehicle together with an identification information given in advance to the vehicle.

11. A driving ability determination method, comprising the steps of:
acquiring time-series travel data of a vehicle;
identifying a driver of the vehicle; and
calculating an evaluation value indicating steering characteristics of the identified driver based on travel data during one cycle from a start time point to an end time point of the vehicle from among the acquired travel data.
